(19) United States
(12) Patent Application Publication
(10) Pub. No.: US 2007/0031366 A1
(43) Pub. Date: Feb. 8, 2007

(54) Process for obtaining a closer shave

(75) Inventors: Alan Meyers, Warren, NJ (US); Isabelle Hansenne, Westfield, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: L'OREAL, Paris (FR)

(21) Appl. No.: 11/199,476

(22) Filed: Aug. 8, 2005

(51) Int. Cl. A61K 8/19

(52) U.S. Cl. 424/73

(57) ABSTRACT

A shaving process for obtaining a closer shave and reducing the subsequent appearance of five o'clock shadow involving the steps of: (a) providing an unshaven skin surface; (b) providing a pre-shave composition containing at least one skin-abrading agent; (c) contacting the unshaven skin surface with the pre-shave composition to form a treated skin surface; (d) removing the pre-shave composition to form a clean, treated skin surface; and (e) shaving the clean, treated skin surface to form a shaved skin surface.

### BACKGROUND OF THE INVENTION

The art of shaving human skin, and particularly mens' faces, is centuries old. Yet the process for shaving is essentially unchanged. The desired result is a smooth skin surface.

One of the drawbacks associated with the daily shaving process is the re-appearance of hair stubble towards the end of the day, also known as “five o'clock shadow.” A five o'clock shadow is unwanted beard growth visible late in the day on a man's shaven face. Thus, as the ‘nine-to-five’ workday ends, the morning's shave grows back.

An object of the present invention is thus to provide a process for shaving a skin surface in order to enhance the closeness of the shave and reduce the subsequent appearance of a five o'clock shadow.

### BRIEF SUMMARY OF THE INVENTION

The present invention generally relates to a process for shaving human skin involving:

providing an unshaven skin surface;

providing a pre-shave composition containing at least one skin-abrading agent;

contacting the unshaven skin surface with the pre-shave composition to form a treated skin surface;

removing the pre-shave composition to form a clean, treated skin surface; and

shaving the clean, treated skin surface to form a shaved skin surface.

### DETAILED DESCRIPTION OF THE INVENTION

Other than in the operating examples, or where otherwise indicated, all numbers expressing quantities of ingredients and/or reaction conditions are to be understood as being modified in all instances by the term “about.”

The present invention is based on the surprising discovery that the use of a pre-shave composition containing at least one skin-abrading agent, prior to shaving, both enhances the closeness of a shave and reduces the subsequent appearance of a five o'clock shadow.

Suitable skin-abrading agents for use in the present invention include, but are not limited to, cellulosic products, such as ground walnut shells, ground apricot kernels, or other plant material (e.g., bark, seed, shell, nut, or combinations thereof), silica, diatomaceous earth, metal oxide, jojoba, pumice, polyethylene, sugar and wax beads.

Metal oxide particles may include magnesium oxide or aluminum oxide. These particles have a purity of at least 95%, better still of at least 99%. Their average particle size preferably ranges from 100 to 180 μm. Aluminum oxide is preferred for use in the present invention, in particular in the anhydrous crystalline form (corundum).

Aluminum oxide particles are available as particles calcined at high temperature, until the α-Al₂O₃ crystalline structure of corundum is obtained, and then treated in order to form grains having sharp edges and having a given particle size distribution, the particles preferably having an average particle diameter of between 100 and 180 μm and preferably between 130 and 150 μm. Their distribution is preferably such that none of the particles has a diameter greater than 250 μm. Such particles are available commercially in particular from MarkeTech International under the trade name Dermagrain™. The particles referenced as Dermagrain™ 900 are composed of crystalline α-alumina with a purity of 99.55% having an average particle size diameter of approximately 140 μm, the particles all having a diameter of less than 250 μm. Less than 3% of the particles have a diameter of less than 105 μm. Other particles are available from Industrial Supply under the trade names ARL 100 and ARL 120. They are aluminum oxide particles having an average particle diameter of 120 and 100 μm respectively and a particle size distribution ranging from 75 to 212 μm and from 63 to 180 μm respectively.

In another embodiment, the metal oxide particles used in the composition according to the invention may be magnesium oxide preferably having an average particle size ranging from 100 to 180 μm. An example of particles of this type is sold by MarkeTech International under the trade name Magnaderm™ 100. They are particles having an average particle diameter of approximately 120 μm and a purity of at least 99%.

In another embodiment the skin-abrading agent used in the pre-shave composition may be ground up fruit kernel powder, such as apricot, peach or nectarine kernel powder, or diatomaceous earth. These powders are available commercially from companies such as Alban Muller.

The skin abrading agent will typically be present in an amount sufficient to abrade the targeted area of skin. Preferably, the skin abrading particles may be present in concentrations ranging from 0.5 to 40%, preferably from 10 to 30% and more preferably from 15 to 25% by weight, based on the total weight of the microdermabrasion composition. This composition preferably has a pH of greater than 4 and less than 8, better still less or equal to 7 and even better still of between 5.5 and 7.

Determining the precise amount and fineness of the skin-abrading agent for a particular formulation is within the skill of the ordinary artisan. In fact, because of the variation in skin types, it is preferable that formulations within the scope of this invention be available in different grades of abrasiveness (e.g., very fine, fine, and medium) so that the user can determine which is optimal for his (or her) own use.

The pre-shave composition may be provided in the form of a lotion, gel, fluid or cream. It may comprise various adjuvants and advantageously may include at least one heterogeneous polysaccharide. This is because the Applicant has found that these compounds make it possible to reduce the discomfort associated with the use of the abovementioned abrasive metal oxide particles. This heterogeneous polysaccharide can be an alginate or alternatively a heterogeneous polysaccharide comprising at least one fucose unit, comprising in particular fucose, galactose and galacturonic acid units, in particular a linear sequence of α-L-fucose, of α-D-galactose and of galacturonic acid. Such a polysaccharide is available in particular in the form of a 1% solution in water from Solabia under the trade name Fucogel 1000 PP®.

The pre-shave composition may be applied to the skin by manual massaging with the fingertips or by mechanical massaging using a vibrating device provided with a massaging head equipped with a pad, as disclosed in Application US2001/0046506 or U.S. Pat. No. 6,652,888, for example.

The at least one skin-abrading agent may be present in any conventional cosmetically- and/or pharmaceutically-acceptable carrier medium.

The pre-shave composition may also include ingredients such as stearic acid, sodium lauryl sulfate, EDTA, and the like to aid in desquamation and exfoliation, as well as other conventionally added ingredients. As mentioned, the amount and fineness of the skin-abrading agent will vary depending upon the abrasive effect desired, and will vary among people because of their differing skin types and facial structures.

Additional ingredients can include medications to combat inflammation and/or infection such as, for example, a glucocorticoid and triclosan.

After the unshaven skin has been treated with the pre-shave composition and cleaned, any conventional skin shaving composition and apparatus (manual or electric razor) may be used.

A skin moisturizing composition and/or a photoprotective composition may also be employed in the process of the present invention. The moisturizer and sunscreen are preferably applied after the skin has been shaved.

The skin moisturizing composition will typically contain at least one skin soothing agent present in an amount sufficient to both soothe previously weakened skin and protect it from further assaults, particularly those from the environment. Suitable skin soothing agents include, but are not limited to, emollients such as silicones, hydrating agents such as polyols, i.e. glycerin and/or propylene glycol which are homogenized together in the presence of an emulsifier, and antioxidants for combating free radicals, such as tocopherol, tocopherol acetate, ascorbic acid and arginine pyrrolidonecarboxylate.

It is highly recommended that a photoprotective composition containing at least one photoprotective agent be applied onto treated skin in order to protect it from the harmful effects of the sun.

Suitable organic photoprotective agents may be chosen from anthranilates; cinnamic derivatives; dibenzoylmethane derivatives; salicylic derivatives; camphor derivatives; triazine derivatives, such as those disclosed in patents and patent applications U.S. Pat. No. 4,367,390, EP863145, EP517104, EP570838, EP796851, EP775698, EP878469, EP933376, EP507691, EP507692, EP790243 or EP944624, the entire contents of which are incorporated herein by reference; benzophenone derivatives; β,β-diphenylacrylate derivatives; benzotriazole derivatives; benzalmalonate derivatives; benzimidazole derivatives; imidazolines; bisbenzoazolyl derivatives, such as disclosed in Patents EP669323 and U.S. Pat. No. 2463264, the entire contents of which are incorporated herein by reference; p-aminobenzoic acid (PABA) derivatives; methylenebis(hydroxyphenylbenzotriazole) derivatives, such as disclosed in patents and patent applications U.S. Pat. No. 5,237,071, U.S. Pat. No. 5,166,355, GB2303549, DE19726184 and EP893119, the entire contents of which are incorporated herein by reference; benzoxazole derivatives, such as disclosed in Patent Applications EP832642, EP1027883, EP1300137 and DE10162844; screening polymers and screening silicones, such as those disclosed in particular in patent application WO93/04665, the entire contents of which are incorporated herein by reference; dimers derived from α-alkylstyrene, such as those disclosed in patent application DE19855649, the entire contents of which are incorporated herein by reference; 4,4-diarylbutadienes, such as disclosed in Applications EP967200, DE19746654, DE19755649, EP1008586, EP1133980 and EP133981, the entire contents of which are incorporated herein by reference, and their mixtures.

The organic photoprotective agents more particularly preferred may be chosen from the following compounds (CTFA names or chemical names):
Ethylhexyl Salicylate,
Ethylhexyl Methoxycinnamate,
Octocrylene,
Phenylbenzimidazole Sulfonic Acid,
Benzophenone-3,
Benzophenone-4,
Benzophenone-5,
4-Methylbenzylidene Camphor,
Terephthalylidene Dicamphor Sulfonic Acid,
Disodium Phenyl Dibenzimidazole Tetrasulfonate,
2,4,6-Tris(diisobutyl 4′-aminobenzalmalonate)-s-triazine,
Anisotriazine,
Ethylhexyl Triazone,
Diethylhexyl Butamido Triazone,
Methylene Bis-Benzotriazolyl Tetramethyl-butylphenol,
Drometrizole Trisiloxane,
Polysilicone-15,
1,1-Dicarboxy (2,2′-dimethylpropyl)-4,4-diphenylbutadiene,
2,4-Bis[5-l(dimethylpropyl)benzoxazol-2-yl-(4-phenyl)imino]-6-(2-ethylhexyl)imino-1,3,5-triazine, and their mixtures.

Suitable inorganic photoprotective agents may be chosen from pigments or alternatively nanopigments (mean size of the primary particles: generally between 5 nm and 100 nm, preferably between 10 nm and 50 nm) formed of metal oxides which may or may not be coated, such as, for example, nanopigments formed of titanium oxide (amorphous or crystalline in the rutile and/or anatase form), iron oxide, zinc oxide, zirconium oxide or cerium oxide, which are all UV photoprotective agents well known per se. Furthermore, conventional coating agents are alumina and/or aluminum stearate. Such nanopigments formed of metal oxides, which may or may not be coated, are disclosed in particular in Patent Applications EP518772 and EP518773, the entire contents of which are incorporated herein by reference.

The photoprotective agents should be present in the photoprotective composition in an amount sufficient to yield an SPF of at least 15.

The various compositions used in the process of the present invention may be provided in the form of: (i) dispersions of the lotion or gel type, (ii) emulsions with a liquid or semi-liquid consistency of the milk type, obtained by dispersion of a fatty phase in an aqueous phase (O/W) or vice versa (W/O), (iii) suspensions or emulsions with a soft, semi-solid or solid consistency of the cream or gel type, (iv) multiple emulsions (W/O/W or O/W/O), (v) microemulsions, (vi) vesicular dispersions of ionic and/or nonionic type, or (vii) wax/aqueous phase dispersions. These compositions are prepared according to methods known to those of ordinary skill in the art of cosmetics or dermatological formulations.

The oils present in these emulsions can be silicone oils, which may be volatile or nonvolatile, hydrocarbon oils or vegetable oils. These emulsions can additionally comprise non-oily fatty substances, such as shea butter, silicone gums, esters of fatty acids and of fatty alcohols, fatty acids and fatty alcohols.

These compositions can additionally comprise various adjuvants commonly used in the cosmetics field, such as emulsifiers, including glyceryl fatty acid esters, sugar fatty acid esters, sorbitan fatty acid esters, polyethylene glycol fatty acid esters, ethoxylated fatty alcohols and alkylpolyglycosides; fillers, in particular polyacrylamide (Nylon) fibers and/or microbeads, silica, optionally in the form of a colloidal dispersion, and/or organic microspheres which are optionally expanded; preservatives and/or copreservatives, such as caprylyl glycol; colorants; fragrances; pH adjusters, such as neutralizing agents and/or buffering agents; ethanol; and thickening and gelling agents, in particular acrylamide homo- and co-polymers, acrylic homo- and co-polymers, acrylamidomethylpropanesulphonic acid (AMPS) homo-and co-polymers, and xanthan gum.

Of course, a person skilled in the art will take care to choose this or these possible additional compounds and/or their amounts so that the advantageous properties of the compositions according to the invention are not, or not substantially, detrimentally affected by the envisaged addition.

The inventive process involves first massaging a cosmetically suitable pre-shave composition containing at least one skin-abrading agent on the face (or other skin area to be shaved) to form a treated skin surface, removing the pre-shave composition to form a clean, treated skin surface and then shaving the clean, treated skin surface using any conventional shaving device.

The process of this invention, although not desirous of being constrained to a particular theory, is believed to enhance the closeness of a shave and thereby reduce the subsequent appearance of a five o'clock shadow most effectively when used on a continuing basis. The continual use provides a constant abrasion to the skin to promote the removal of dead skin cells and newly grown skin lying over hairs on the skin surface.

According to another embodiment of the present invention, there is provided a kit containing: (a) a multi-unit receptacle; (b) a unit having a pre-shave composition containing at least one skin-abrading agent; (c) a unit having a skin shaving composition; (d) optionally, a unit having a skin moisturizing composition; and (e) optionally, a unit having a photoprotective composition.

The present invention will be better understood from the examples which follow, all of which are intended for illustrative purposes only and are not meant to unduly limit the scope of the invention in any way.

### EXAMPLES

A consumer test was performed in order to determine the effectiveness of the process of the present invention. A pre-shave composition was prepared having the following formulation:

| | |
|---|---|
| BHT | 0.025 |
| CETEARETH-60 MYRISTYL GLYCOL | 0.5 |
| DISODIUM EDTA | 0.26 |
| DISODIUM RICINOLEAMIDO MEA-SULFOSUCCINATE | 8.5 |
| FRAGRANCE | 0.4 |
| GLYCERIN | 1 |
| LAURIC ACID | 2.25 |
| MAGNESIUM ALUMINUM SILICATE | 0.5 |
| METHYLPARABEN | 0.2 |
| MYRISTIC ACID | 9 |
| PALMITIC ACID | 7.2 |
| PEG-7 GLYCERYL COCOATE | 2 |
| PHENOXYETHANOL | 0.7 |
| POLYETHYLENE | 10 |
| POTASSIUM HYDROXIDE | 6.8 |
| PROPYLPARABEN | 0.1 |
| PRUNUS ARMENIACA (APRICOT) SEED POWDER | 2 |
| STEARIC ACID | 3.6 |
| TOCOPHEROL (and) GLYCINE SOJA (SOYBEAN) OIL | 0.1 |
| WATER | 45.765 |

8 Caucasian males ages 26-61, who shaved at least 5 times a week, treated half of their unshaven face with the above-disclosed pre-shave composition prior to shaving leaving the other half untreated. After treatment and removal of the pre-shave composition, each male shaved his face using a razor. 6 out of 8 males felt the shave they obtained on the treated side of their face was closer than that on the untreated side. Moreover, 7 out of 8 males felt that the appearance of a five o'clock shadow (8 hours after shaving) was less pronounced on the treated side of their face.

Yet another embodiment of pre-shave composition which may be used according to the process of the present invention corresponds to the following formulation:

| | |
|---|---|
| DISODIUM EDTA | 0.05 |
| ALUMINA | 20 |
| MINERAL OIL | 10 |
| BUTYROSPERMUM PARKII (SHEA BUTTER) | 2 |
| AMMONIUM POLYACRYLOYLDIMETHYL TAURATE | 0.8 |
| DIMETHICONE | 2 |
| WATER | 55.65 |
| GLYCERIN | 5 |
| GLYCERYL STEARATE (and) PEG-100 STEARATE | 3 |
| STEARYL ALCOHOL | 1.5 |

What is claimed is:

1. A process for shaving human skin comprising:
(a) providing an unshaven skin surface;
(b) providing a pre-shave composition containing at least one skin-abrading agent;
(c) contacting the unshaven skin surface with the pre-shave composition to form a treated skin surface;
(d) removing the pre-shave composition to form a clean, treated skin surface; and
(e) shaving the clean, treated skin surface to form a shaved skin surface,
wherein the process yields a closer shave and a reduction in appearance of five o'clock shadow.

2. The process of claim 1 wherein the at least one skin-abrading agent is chosen from metal oxide, polyethylene, pumice, ground plant material, diatomaceous earth, silica, waxes and sugar.

3. The process of claim 1 further comprising contacting the shaved skin surface with at least one composition chosen from a moisturizing composition and a photoprotective composition.

4. The process of claim 3 wherein the photoprotective composition has an SPF of at least about 15.

5. The process of claim 1 wherein the process is conducted on a daily basis.

6. The process of claim 1 wherein the process is conducted on alternate days.

7. The process of claim 1 wherein the at least one skin-abrading agent is aluminum oxide.

8. The process of claim 1 wherein the at least one skin-abrading agent is a ground plant material.

9. The process of claim 1 wherein the at least one skin-abrading agent is polyethylene.

10. A kit comprising:
(a) a multi-unit receptacle;
(b) a unit containing a pre-shave composition having at least one skin-abrading agent;
(c) a unit containing a skin shaving composition;
(d) optionally, a unit containing a moisturizing composition; and
(e) optionally, a unit containing a photoprotective composition.

11. The kit of claim 10 wherein the moisturizing and/or photoprotective composition has an SPF of at least about 15.

12. The kit of claim 10 wherein the at least one skin-abrading agent is aluminum oxide.

13. The kit of claim 10 wherein the at least one skin-abrading agent is a ground plant material.

14. The kit of claim 10 wherein the at least one skin-abrading agent is polyethylene.
